(19) United States
(12) Patent Application Publication
(10) Pub. No.: US 2025/0022605 A1
(43) Pub. Date: Jan. 16, 2025

(54) METHOD AND COMPUTING DEVICE FOR INDENTIFYING LATENT OBESITY

(71) Applicant: Endominance, Inc., McLean, VA (US)

(72) Inventors: Paul Yeon LEE, McLean, VA (US); Jin Hoon AN, Seoul (KR)

(73) Assignee: Endominance, Inc., McLean, VA (US)

(21) Appl. No.: 18/608,236

(22) Filed: Mar. 18, 2024

Related U.S. Application Data
(60) Provisional application No. 63/526,002, filed on Jul. 11, 2023.

(51) Int. Cl. G16H 50/30; G16H 10/20

(52) U.S. Cl. CPC G16H 50/30; G16H 10/20

(57) ABSTRACT

The present disclosure relates to a method for identifying latent obesity, and may include the steps of providing a user with an orientation assessment questionnaire configured to measure a plurality of independent orientation parameters, including perception orientation, conception orientation, and behavioral orientation; providing the user with an obesity assessment questionnaire configured to measure health characteristics, including personal health history, current health, and awareness of personal health; receiving user input in response to the orientation assessment questionnaire and the obesity assessment questionnaire; for each of the independent orientation parameters, computing a corresponding orientation metric according to the user input in response to the orientation assessment questionnaire; for each of a plurality of health characteristics, computing a corresponding health metric according to the user input in response to the obesity assessment questionnaire; in accordance with the computed orientation metrics and the computed health metrics, determining a latent obesity type for the user and an obesity probability projection for the user; in accordance with the determined latent obesity type and the obesity probability projection, generating an obesity mitigation action plan with a plurality of steps; and displaying the action plan on a display.

### CROSS REFERENCE TO RELATED APPLICATION

The present application claims priority to U.S. Provisional Patent Application No. 63/526,002, filed Jul. 11, 2023, the entire contents of which is incorporated herein for all purposes by this reference.

### TECHNICAL FIELD

The present disclosure relates to a method and computing device for identifying latent obesity

### BACKGROUND

Conventional obesity measurement and personalized solutions mainly focus on the physical aspect of obesity and are limited to providing solutions for this aspect. Further, there is no proven method for measuring how obesity is occurred by an individual's lifestyle or behavioral habits.

This solution is based on the user's physical attributes (degree of obesity, age, gender, physical ability, disease and disability, etc.) and presents only the resulting symptoms (degree of obesity) that cause obesity. Therefore, it is difficult to fully understand the causes of obesity.

Moreover, in the currently provided solution, in the case where two people of the same age and gender show similar symptoms (degree of obesity, physical ability, level of participation, etc.), they are regarded as the same person from the point of view of obesity, and provided with the same solution. Due to this, the success rate of a number of personalized exercise programs, diet programs, and obesity treatment programs is declining.

Therefore, providing personalized solutions based solely on body fat metric is equivalent to prescribing medication based solely on symptoms without a diagnosis. For this reason, current personalized solutions do not achieve individualization and provide general solutions biased only for symptoms.

### SUMMARY OF THE DISCLOSURE

The present disclosure relates to a method and computing device for identifying latent obesity to provide a user-customized obesity solution.

More specifically, the present disclosure relates to a method and computing device for identifying latent obesity to provide a user-customized obesity solution in consideration of a user's lifestyle, behavioral pattern, and cognitive orientation.

According to the present disclosure, a method for identifying latent obesity may comprise, at a computing device having a display, one or more processors, and memory storing one or more programs configured for execution by the one or more processors which provide a user with an orientation assessment questionnaire configured to measure a plurality of independent orientation parameters, including perception orientation, conception orientation, and behavioral orientation, provide the user with an obesity assessment questionnaire configured to measure health characteristics, including personal health history, current health, and awareness of personal health, receive user input in response to the orientation assessment questionnaire and the obesity assessment questionnaire, for each of the independent orientation parameters, compute a corresponding orientation metric according to the user input in response to the orientation assessment questionnaire, for each of a plurality of health characteristics, compute a corresponding health metric according to the user input in response to the obesity assessment questionnaire, in accordance with the computed orientation metrics and the computed health metrics, determine a latent obesity type for the user and an obesity probability projection for the user, in accordance with the determined latent obesity type and the obesity probability projection, generate an obesity mitigation action plan with a plurality of steps, and display the action plan on the display.

Further, the health characteristics may further include current physical environment and current lifestyle of the user.

Further, the method may further include determining one or more obesity inducing stress markers for the user, in accordance with the computed orientation metrics and a first set of the computed health metrics corresponding to the current physical environment and current lifestyle of the user.

Further, generating the obesity mitigation action plan includes utilizing the determined obesity inducing stress markers for the user.

Further, the method may further comprise determining an obesity incidence precursor projection for the user, in accordance with the determined obesity inducing stress markers and a second set of the computed health metrics corresponding to the personal health history and the awareness of personal health wherein the obesity probability projection for the user is further determined based on the determined obesity incidence precursor projection.

Further, the determined obesity probability projection may represent a probability of whether obesity will occur within a predetermined span of time.

Further, at least one of the plurality of steps may include receiving user feedback in response to the action plan.

Further, at least one of the plurality of steps may include providing exercise instructions for the user to follow.

Further, the latent obesity type may belong to a category that represents a particular physical engagement level, a particular cognitive engagement level, and a particular behavioral engagement level derived from the computed orientation metrics.

Meanwhile, according to the present disclosure, a computing device may comprise one or more processors, memory, and one or more programs stored in the memory and configured for execution by the one or more processors, the one or more programs including instructions for providing a user with an orientation assessment questionnaire configured to measure a plurality of independent orientation parameters, including perception orientation, conception orientation, and behavioral orientation, providing the user with an obesity assessment questionnaire configured to measure health characteristics, including personal health history, current health, and awareness of personal health, receiving user input in response to the orientation assessment questionnaire and the obesity assessment questionnaire, for each of the independent orientation parameters, computing a corresponding orientation metric according to the user input in response to the orientation assessment questionnaire, for each of a plurality of health characteristics, computing a corresponding health metric according to the user input in response to the obesity assessment questionnaire, in accordance with the computed orientation metrics and the computed health metrics, determining a latent obesity type for the user and an obesity probability projection for the user, in accordance with the determined latent obesity type and the obesity probability projection, generating an obesity mitigation action plan with a plurality of steps; wherein the computing device further comprise a display configured to display the action plan on the display.

Further, the health characteristics may further include current physical environment and current lifestyle of the user.

Further, the one or more programs may further include instructions for, in accordance with the computed orientation metrics and a first set of the computed health metrics corresponding to the current physical environment and current lifestyle of the user, determining one or more obesity inducing stress markers for the user.

Further, generating the obesity mitigation action plan may include utilizing the determined obesity inducing stress markers for the user.

Further, the one or more programs may further include instructions for, in accordance with the determined obesity inducing stress markers and a second set of the computed health metrics corresponding to the personal health history and the awareness of personal health, determining an obesity incidence precursor projection for the user, wherein the obesity probability projection for the user may be further determined based on the determined obesity incidence precursor projection.

Further, the determined obesity probability projection may represent a probability of whether obesity will occur within a predetermined span of time and at some points in the future.

Further, at least one of the plurality of steps may include receiving user feedback in response to the action plan.

Further, at least one of the plurality of steps may include providing exercise instructions for the user to follow.

Further, the latent obesity type may belong to a category that represents a particular physical engagement level, a particular cognitive engagement level, and a particular behavioral engagement level derived from the computed orientation metrics.

The method for identifying latent obesity according to the present disclosure may provide a user with an orientation assessment questionnaire configured to measure a plurality of independent orientation parameters including perception orientation, conception orientation, and behavior orientation. Through this, the present disclosure may provide a new and improved customized obesity solution by defining a user's obesity type based on the user's cognitive and behavioral orientations, unlike the method of specifying the user's obesity type based only on the user's body attributes (age, weight, etc.).

Furthermore, the method for identifying latent obesity according to the present disclosure may provide the user with an obesity assessment questionnaire configured to measure health characteristics including personal health history, current health, and awareness of personal health. Through this, the present disclosure may determine the user's obesity type by considering a wider range of factors, including the user's physical condition, lifestyle, and life patterns, beyond the method of determining the user's obesity type based on quantitative body values.

Furthermore, the method for identifying latent obesity according to the present disclosure may calculate a corresponding orientation metric based on user responses to the orientation assessment questionnaire, calculate a corresponding health metric based on user responses to the obesity assessment questionnaire, and determine a latent obesity type and an obesity probability projection for the user using the orientation metric and the health metric. Through this, the present disclosure may determine the user's obesity type in a systematic and subdivided manner.

Furthermore, the method for identifying latent obesity according to the present disclosure may provide the user with a customized solution for items to be improved by the user for potential future health risks by providing an obesity mitigation action plan with a plurality of steps in accordance with the latent obesity type and the obesity probability projection.

### BRIEF DESCRIPTION OF THE DRAWINGS

FIGS. 1, 2 and 3 are conceptual diagrams for explaining a computing device for identifying latent obesity according to the present disclosure.

FIG. 4 is a flow chart for explaining a method for identifying latent obesity according to the present disclosure.

FIGS. 5 and 6 are conceptual diagrams for explaining a questionnaire process performed by the present disclosure.

FIG. 7 is a conceptual diagram for explaining orientation assessment according to the present disclosure.

FIGS. 8A and 8B are conceptual diagrams for explaining an obesity type algorithm according to the present disclosure.

FIG. 9 is a conceptual diagram for explaining obesity assessment according to the present disclosure.

FIGS. 10, 11, 12, and 13 are conceptual diagrams for explaining algorithms for deriving an obesity solution.

FIGS. 14 and 15 are conceptual diagrams for explaining an obesity solution provided by the present disclosure.

### DETAILED DESCRIPTION OF THE DISCLOSURE

Description will now be given in detail according to exemplary embodiments disclosed herein, with reference to the accompanying drawings. For the sake of brief description with reference to the drawings, the same or equivalent components may be provided with the same or similar reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In the present disclosure, that which is well-known to one of ordinary skill in the relevant art has generally been omitted for the sake of brevity. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

It will be understood that although the terms first, second, etc. may be used herein to describe various elements, these elements should not be limited by these terms. These terms are generally only used to distinguish one element from another.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

A singular representation may include a plural representation unless it represents a definitely different meaning from the context.

In the present disclosure, the terms such as “include” and/or “have” may be construed to denote a certain characteristic, number, step, operation, constituent element, component or a combination thereof, but may not be construed to exclude the existence of or a possibility of addition of one or more other characteristics, numbers, steps, operations, constituent elements, components or combinations thereof.

The present disclosure may define the type of obesity associated with the user's current physical condition, environmental state, and cognitive behavioral orientation, and may provide a new and improved customized solution for obesity.

In the present disclosure, the physical condition may refer to an individual's physical characteristics and health condition. For example, as some factors of physical condition, there may be weight, body fat content, body composition, and the like. As other factors, there may be blood pressure, blood sugar, cholesterol level, cardiovascular health, strength, flexibility, and the like.

Furthermore, the environmental state may refer to the surrounding environment and external factors in which the individual lives. For example, it may include the place and surroundings where an individual lives, social culture, work environment, relationships with family members, eating habits, life patterns, social support systems, and the like.

Meanwhile, the cognitive behavioral orientation in the present disclosure may mean the orientation related to proactive and selective identification of external factors that appear in the process of recognizing a specific situation and determining and performing actions for the previously recognized situation based on a pre-existing concept. In other words, assuming that the external environment is a set of fixed objects, what to see among them (perception/perceived stimuli) may mean a self-directed identification behavior that proceeds differently for each user (person) according to the user's own subjective orientation.

Such cognitive behavioral orientation may be classified into a plurality of domains related to human cognition and behavior. For example, the cognitive-behavioral orientation may include at least one of a perception domain, a conception domain, a physical aptness domain, an interpersonal domain, a self-directedness domain, and a task execution domain.

The perception domain may refer to a range occupied by a process of perceiving a surrounding situation through senses such as sight among the cognitive behavioral orientation. In this case, the perception may be replaced with terms such as recognition, cognition, sight, awareness, and understanding.

The conception domain may refer to a range occupied by a process in which a person makes a judgment on a perceived situation among the cognitive behavioral orientation. In this case, the conception may be replaced with terms such as interpretation, plan, idea, disclosure, thought, and design.

The physical aptness domain may refer to a scheme and range of using the body in a process of implementing a judgment result on a situation, among the cognitive behavioral orientation. In this case, physical aptness may be replaced with terms such as external activity amount, activity orientation, movement orientation, and body momentum.

The interpersonal domain may refer to a scheme and range in which a person considers other people in a process of performing a judgment result on a situation, among the cognitive behavioral orientation.

The self-directedness domain may refer to a scheme and range of arbitrary exercise of the leading decision-making right, whether a person is more influenced by the outside in decision-making or, on the contrary, a person has an orientation to make a decision on his or her own initiative and expand it to the outside, among the cognitive behavioral orientation. In this case, the self-directedness may be replaced with terms such as leadership, challenge, and adventurousness.

The task execution domain may refer to a scheme and range of whether a person actually implements a result of a judgment on a situation, among the cognitive behavioral orientation. In this case, the task execution may be replaced with terms such as task performance and work execution.

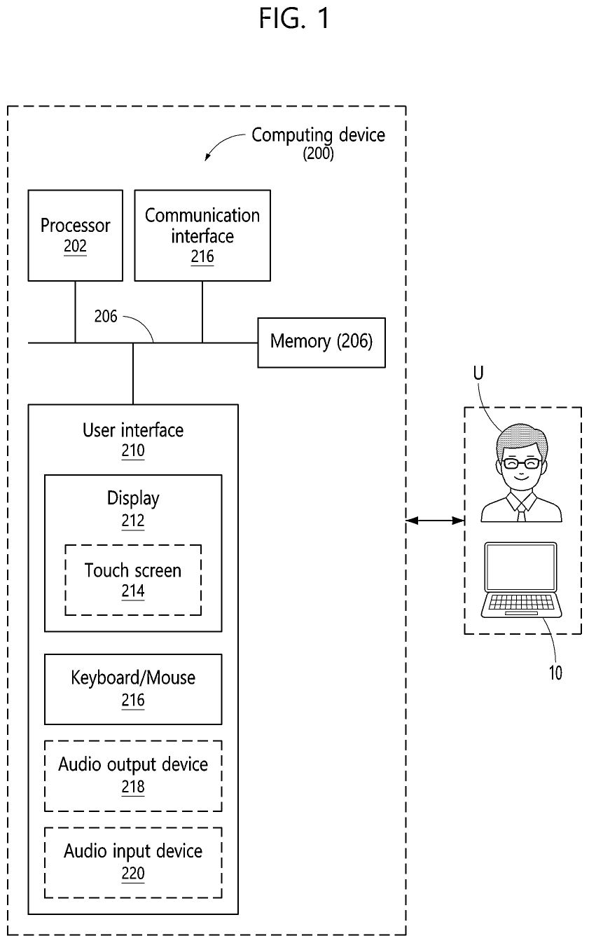

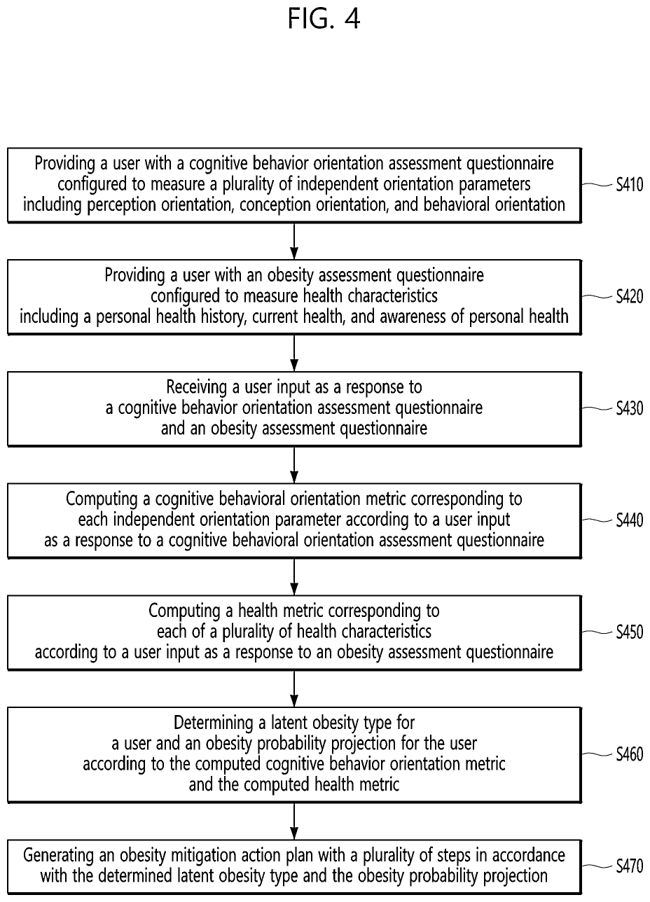

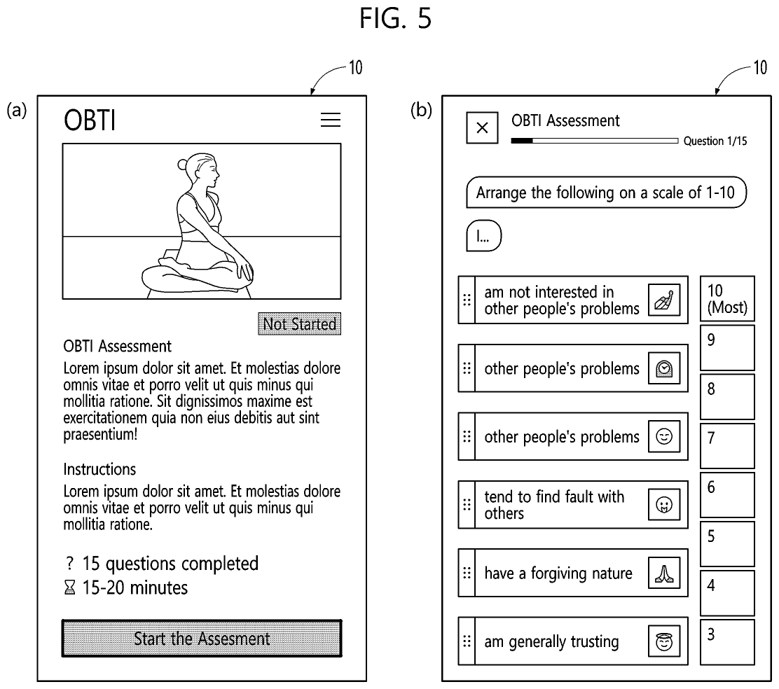

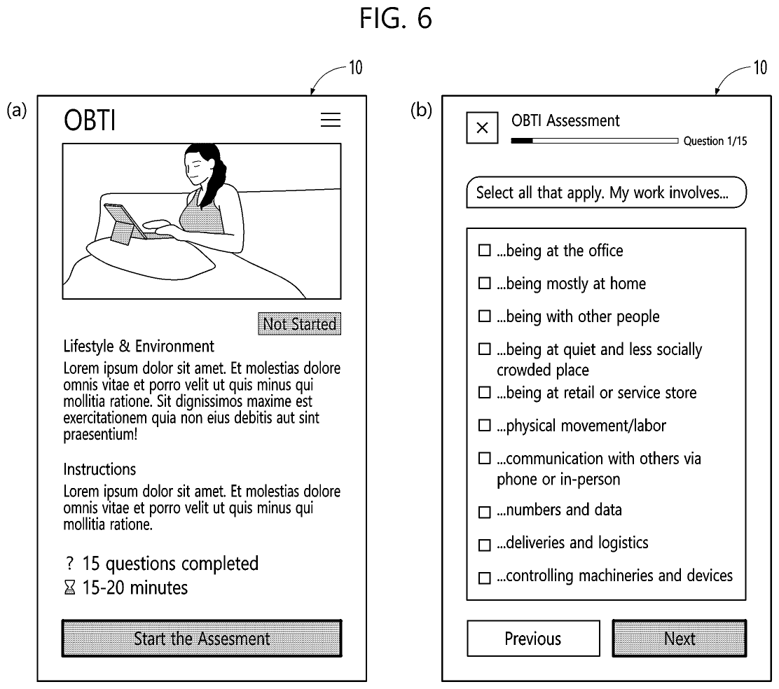

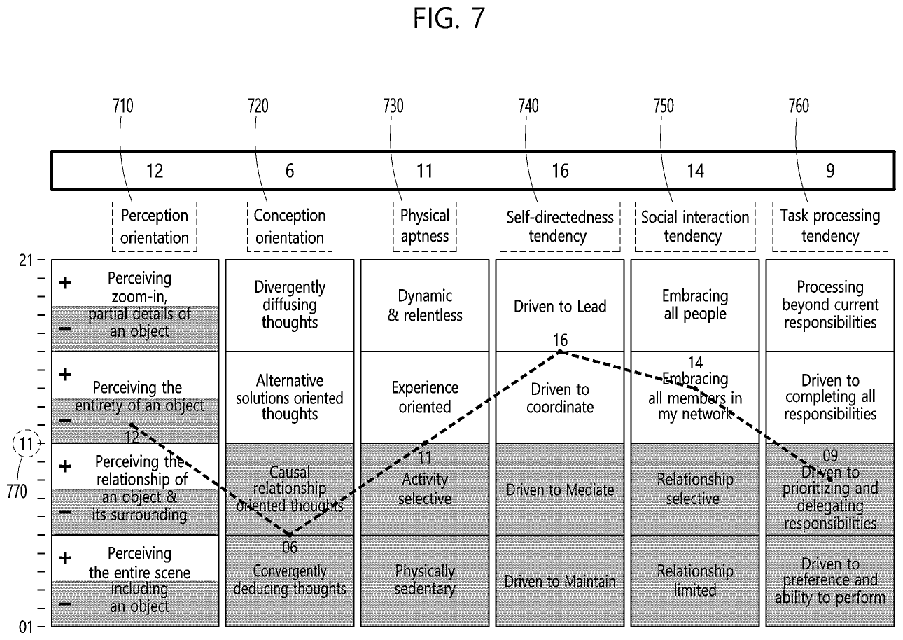

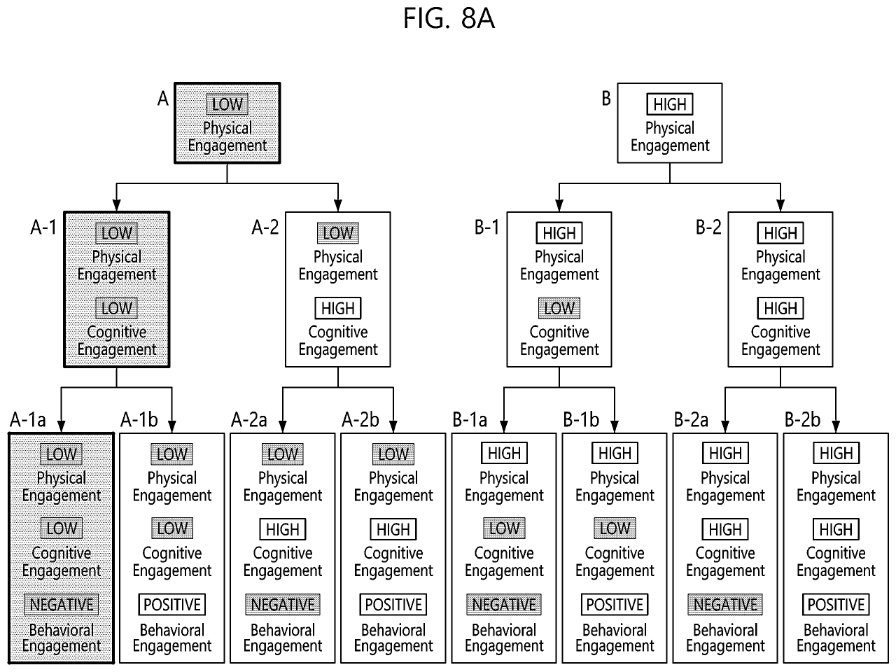

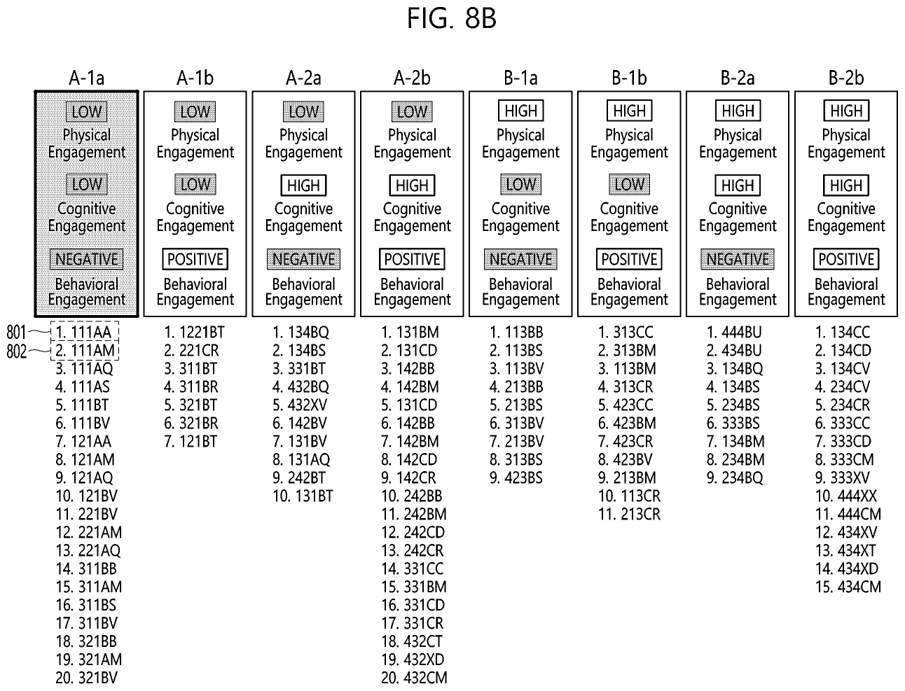

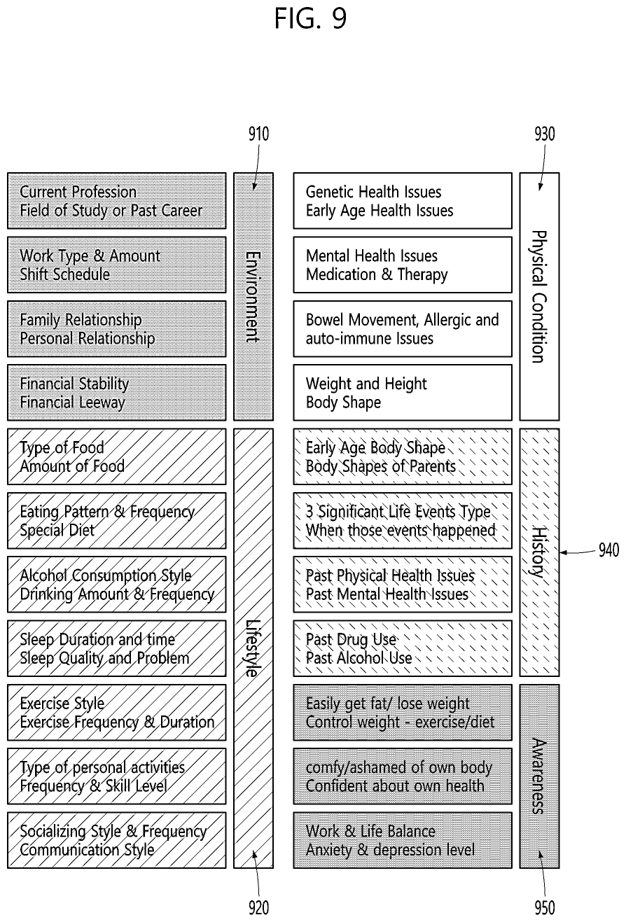

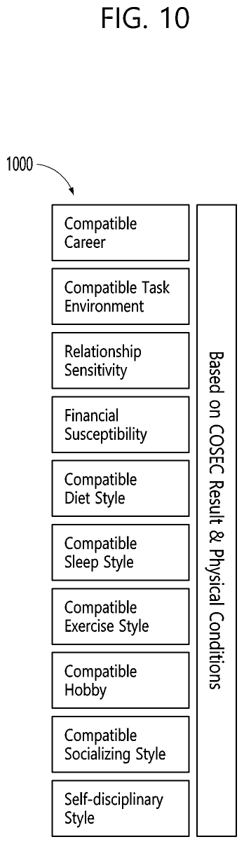

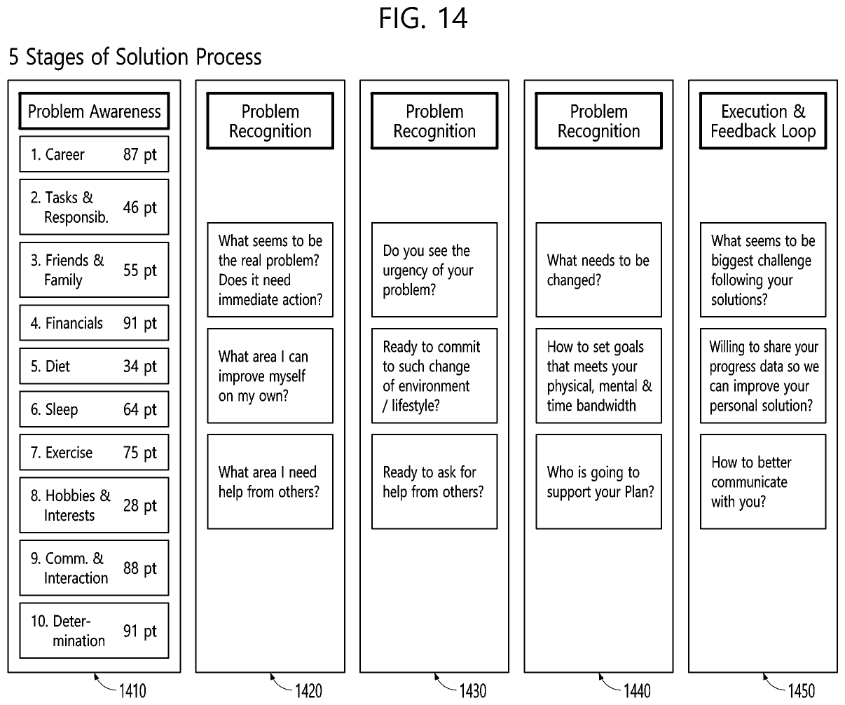

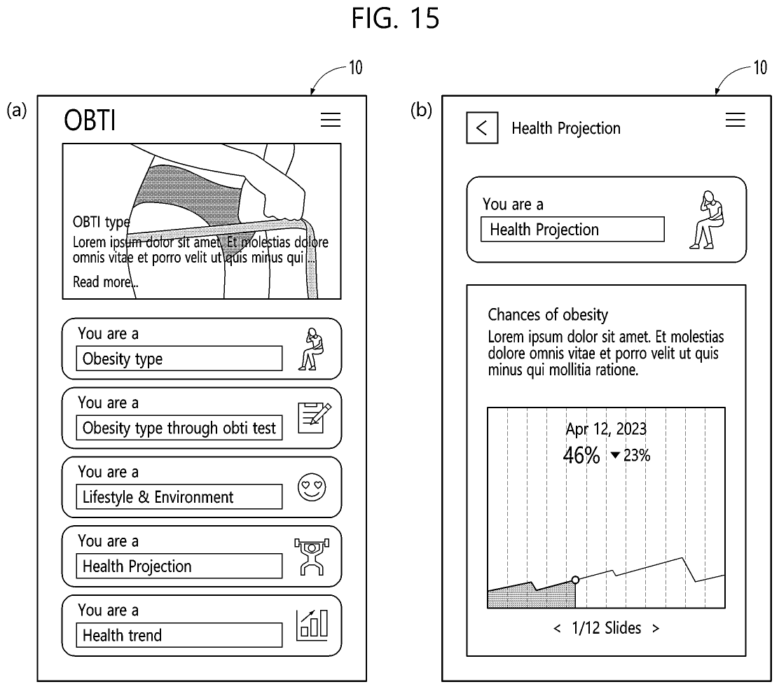

FIGS. 1, 2 and 3 are conceptual diagrams for explaining a computing device for identifying latent obesity according to the present disclosure. FIG. 4 is a flow chart for explaining a method for identifying latent obesity according to the present disclosure. FIGS. 5 and 6 are conceptual diagrams for explaining a questionnaire process performed by the present disclosure. FIG. 7 is a conceptual diagram for explaining orientation assessment according to the present disclosure. FIGS. 8A and 8B are conceptual diagrams for explaining an obesity type algorithm according to the present disclosure. FIG. 9 is a conceptual diagram for explaining obesity assessment according to the present disclosure. FIGS. 10, 11, 12, and 13 are conceptual diagrams for explaining algorithms for deriving an obesity solution. FIGS. 14 and 15 are conceptual diagrams for explaining an obesity solution provided by the present disclosure.

As shown in FIG. 1, a computing device 200 according to the present disclosure may operate in association with a user terminal 10. In this case, the computing device (or computing apparatus, 200) may also be named an “obesity identification system 200”.

The user terminal 10 may include at least one of a mobile phone, a smart phone, a notebook computer, a laptop computer, a slate PC, a tablet PC, an ultrabook, a desktop computer, a digital broadcast terminal, a personal digital assistant (PDA), a portable multimedia player (PMP), a navigator, and a wearable device (for example, a smart watch, smart glasses, a head mounted display (HMD)).

Meanwhile, the computing device 200 according to the present disclosure is not limited to an application, software, or a website, and may be implemented in various ways.

According to the software implementation of the computing device 200 according to the present disclosure, embodiments such as procedures and functions described in the present disclosure may be implemented as separate software modules. Each of the software modules may perform one or more functions and operations described herein.

In this way, the computing device 200 implemented as an application is downloaded through a program capable of downloading applications on the user terminal 10 (e.g., Play Store or App Store), or may be implemented through an initial installation program on the user terminal. In this case, at least one of the processor 202, communication interface 204, memory 206, and user interface 210 of the computing device 200 according to the present disclosure may be understood as a component of the user terminal 10.

Meanwhile, the computing device 200 may exist inside a server (hereinafter referred to as a “server”) built to perform a specific purpose (e.g., obesity projection) separately from the user terminal 10, or may exist as a separate device from the server. In the case where the computing device 200 exists inside the server, the computing device 200 according to the present disclosure may provide a latent obesity projection service through at least one component of the processor 202, communication interface 204, memory 206, user interface 210 located inside the server, or through a module that performs a function similar to each of the above components.

Meanwhile, as shown in FIG. 1, the computing device 200 according to the present disclosure may include at least one of the processor 202, the communication interface 204, the memory 206, and the user interface 210. The above components are software components, and may perform functions in conjunction with hardware components of the user terminal 10.

The user interface 210 may provide a function for exchanging information between a user and the computing device 200. The user interface 210 may include at least one of a display 212, keyboard/mouse 216, audio output device 218, and audio input device 220 provided in the user terminal 10. In the case where the display 212 is a touch screen 214, it can provide both information output and input functions.

The user interface 210 may receive a user input for at least one questionnaire included in the cognitive behavioral orientation test and the obesity projection test. For example, the user interface 210 may select at least one option included in the questionnaire. Here, “to be selected” may mean that when a user's selection is made on a user interface (e.g., the touch screen, 214), a selection signal (or an input signal, or a user input) corresponding to the user's selection is received.

Next, the communication interface 204 may be configured to communicate with the user terminal 10 possessed by the user (or a target user). In the case where communication is made between the user terminal 10 and the communication interface 204, the computing device 200 may receive responses to questionnaires related to cognitive behavioral orientation and obesity projection tests input from the user terminal 10 through the communication interface 204.

The communication interface 204 may support various communication schemes according to the communication standards of communicating devices.

For example, the communication interface 204 may be configured to perform communications using at least one Wireless LAN (WLAN), Wireless-Fidelity (Wi-Fi), Wireless Fidelity (Wi-Fi) Direct, Digital Living Network Alliance (DLNA), Wireless Broadband (WiBro), World Interoperability for Microwave Access (WiMAX), High Speed Downlink Packet Access (HSDPA), High Speed Uplink Packet Access (HSUPA), Long Term Evolution (LTE), Long Term Evolution-Advanced (LTE-A), 5th Generation (5G) Mobile Telecommunication, Bluetooth™ Radio Frequency Identification (RFID), Infrared Data Association (IrDA), Ultra-Wideband (UWB), ZigBee, Near Field Communication (NFC), Wi-Fi Direct, Wireless Universal Serial Bus (USB) technologies.

Next, the memory 206 may store data and instructions necessary for the operation of the computing device 200 according to the present disclosure.

In addition, the memory 206 may store questionnaires related to the cognitive behavioral orientation and obesity projection test. In this case, each of a plurality of items included in the questionnaire and each of a plurality of domains may be matched and stored in the memory 206.

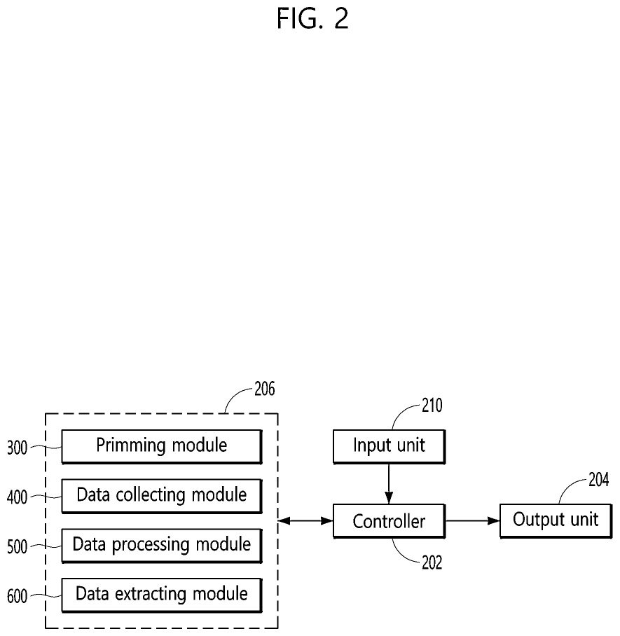

Meanwhile, referring to FIG. 2, the memory 206 may store a priming module 300, a data collecting module 400, a data processing module 500, and a data extracting module 600.

Accordingly, each of the modules 300, 400, 500, and 600 stored in the memory 206 may be controlled by the processor 202, and in this case, the processor 202 may collect and process the data input through the user interface 210 using each module (300, 400, 500, 600) in the memory 206, or may output the collected and processed data through the communication interface 204. In some implementations, the processor 202 may be configured as a hardware device implemented by various electronic circuits (e.g., computer, microprocessor, CPU, ASIC, circuitry, logic circuits, etc.) and be configured to execute instructions stored in the memory 206 to cause the processor 202 provide the functionalities of modules 300, 400, 500 and 600. Herein, the processor 202 and the memory 206 may be implemented as separate semiconductor circuits. Alternatively, the processor 202 and the memory 206 may be implemented as a single integrated semiconductor circuit. The processor 202 may embody one or more processor(s).

Meanwhile, in the present disclosure, tests for cognitive behavioral orientation and obesity are performed, respectively, and test results for cognitive behavioral orientation and obesity are combined to determine the latent obesity type and obesity probability projection for the user. The test order for each of the cognitive behavioral orientation and obesity may be variously performed. After the cognitive behavioral orientation test is performed, the obesity test may be performed. Alternatively, after the obesity test is performed, the cognitive behavioral orientation test may be performed. Alternatively, it is possible to simultaneously perform tests for cognitive behavioral orientation and obesity. However, in the present disclosure, for convenience of explanation, it will be described that the obesity test is performed after the test for cognitive behavioral orientation is performed.

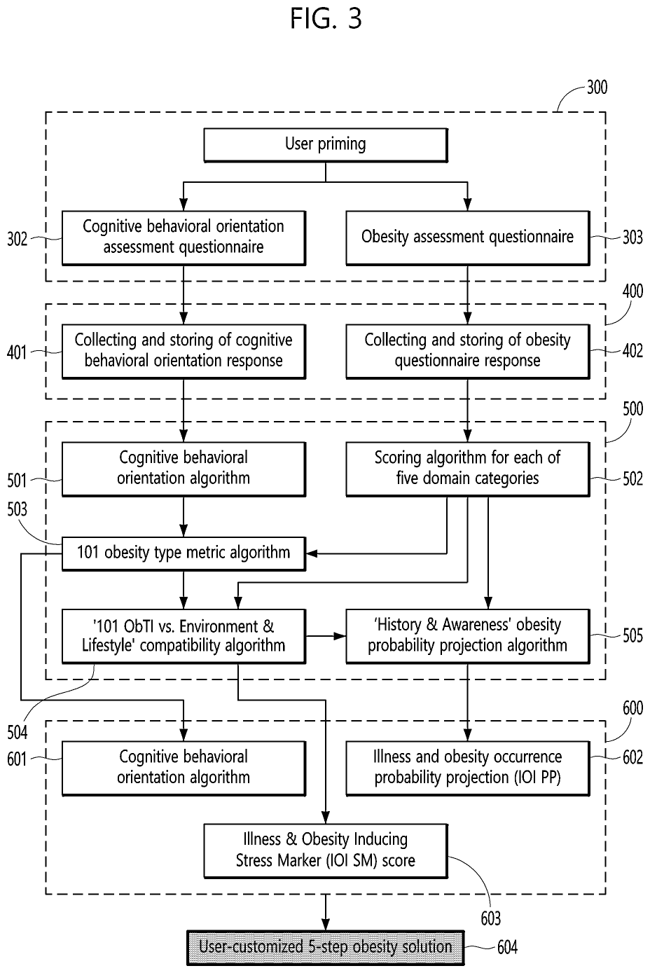

As shown in FIG. 3, the priming module 300 may perform user priming 301 in relation to the current mental state of the user U. Here, priming may mean an operation to stabilize the current mental state of the user U. To this end, the processor 202 may identify the current psychological state of the user U through the priming module 300, and may perform the priming for the cognitive behavioral orientation (or obesity) test or stop testing for the cognitive behavior orientation (or obesity), based on the identified current psychological state.

The processor 202 may request a priming response through the communication interface 204 and receive a priming response through the user interface 210 in response thereto. Accordingly, the processor 202 may determine whether or not to perform the cognitive-behavioral orientation test based on the input priming response, and may perform the user priming 301 by performing a preset priming processor such as inducing the user U to take a deep breath through the priming module 300.

In addition, when receiving input on whether the psychological state of the user (U) is suitable for performing a test on cognitive behavioral orientation, as a priming response to the priming response request, the processor 202 may output to the user terminal 10 a questionnaire (e.g., OSEC Assessment, 302) related to the test for cognitive behavioral orientation if the input indicates that it is suitable for performing the cognitive behavioral orientation test according to the priming response.

For another example, as a priming response, if the user input inputted by the user U indicates that it is not suitable for performing the cognitive-behavioral orientation test (or an obesity test), the processor 202 may output a screen prepared to receive input of remind information, along with the message “Please select a test schedule”. Here, the reminder information may be information set to inform the user U of a notification message set to perform a test related to cognitive behavioral orientation (or obesity) through the user terminal 10. The remind information may include date information, time information, notification means information, and the like.

Further, when receiving a response to whether or not the cognitive behavioral orientation test has been completed from the user terminal 10, the processor 202 may output to the user terminal 10 an obesity assessment questionnaire (Unique Obesity Questionnaire, 303) uniquely configured to measure health characteristics.

The data collecting module 400 may include a first data collecting module 401 that collects and stores responses of the user U input into the cognitive behavioral orientation assessment questionnaire and a second data collecting module 402 that collects and stores responses of the user U input into the obesity assessment questionnaire.

The processor 202 may collect and store responses of the user U input to the cognitive behavioral orientation assessment questionnaire and obesity assessment questionnaire through the data collecting module 400.

The data processing module 500 may include a plurality of different algorithms 501 to 505 for generating result data and content for user responses stored in the data collecting module 400. In the present disclosure, a latent obesity identification service may be provided based on at least one of a plurality of algorithms 501 to 505.

A first algorithm among the plurality of algorithms 501 to 505 may be named a cognitive behavioral orientation algorithm (e.g., COSEC Algorithm, 501). The cognitive behavioral orientation algorithm 501 may be configured to compute a directedness metric (which may also be referred to as “score”) for each of a plurality of preset directedness parameters, based on the user response to an orientation assessment questionnaire for the cognitive behavioral orientation assessment. Meanwhile, in the present disclosure, the “cognitive behavioral orientation” may be used interchangeably with “directedness”.

A plurality of directedness parameters may be predefined. For example, as shown in FIG. 7, the plurality of directedness parameters may include at least one of i) perception orientation 710, ii) conception orientation 720, iii) physical aptness orientation 730, iv) self-directedness tendency 740, v) social interaction tendency 750, and vi) task processing tendency 760.

In the present disclosure, a group including at least one directedness parameter may be referred to as a “domain (or region)”. A group including the parameters of perception orientation 710, conception orientation 720, and physical aptness orientation 730 may be named “cognitive orientation” or “physical aptness domain”. In addition, a group including parameters of self-directedness tendency 740, social interaction tendency 750, and task processing tendency 760 is named “behavioral tendencies” or “preferences domain”. In addition, in the present disclosure, domains and parameters may be used interchangeably. For example, the behavioral tendencies domain may also be named and described as a behavioral tendencies parameter.

The data processing module 500 may calculate a directedness metric corresponding to each of a plurality of directedness parameters based on the cognitive behavioral orientation algorithm 501.

A second algorithm is an algorithm that calculates the user's health metric based on the response to the obesity questionnaire, and may be understood as a scoring algorithm for each of five domain categories (hereinafter referred to as a scoring algorithm, 502). The scoring algorithm 502 may be configured to calculate a health metric (also referred to as “score”) for each of a plurality of predefined health characteristics based on user responses to an obesity assessment questionnaire.

A plurality of health characteristics may be predefined. For example, as shown in FIG. 9, the plurality of health characteristics in the present disclosure may include at least one of the user's current or past i) physical environment (or physical residential environment) 910, ii) lifestyle 920, iii) physical condition 930, iv) personal health history 940, v) personal health awareness 950.

The data processing module 500 may calculate a health metric corresponding to each of a plurality of health characteristics based on the scoring algorithm 502.

A third algorithm is an algorithm for specifying the user's obesity type based on a combination of at least some of the directedness metric and the health metric, and may be named an obesity type algorithm (or 101 obesity type metric algorithm, 503). The processor 202 may specify a latent obesity type of the user based on the obesity type algorithm 503. In this case, the specified latent obesity type may belong to a category representing a specific body engagement level, a specific cognitive engagement level, and a specific behavioral engagement level derived from the directedness metric.

A fourth algorithm may be understood as an algorithm for deriving at least one obesity inducing marker that causes the user's obesity based on information corresponding to the user's latent obesity type and information compatible (or corresponding) among health metrics corresponding to the environment and lifestyle. In the present disclosure, the fourth algorithm is “‘101 ObTI vs. Environment & Lifestyle’ compatibility algorithm 504”. The data processing module 500 may specify at least one obesity inducing marker that causes the user's obesity, based on information corresponding to the user's latent obesity type and information compatible among the user's health metrics (in particular, health metrics for environmental health characteristics and lifestyle health characteristics), according to the fourth algorithm.

A fifth algorithm may be understood as an obesity probability projection algorithm (‘History & Awareness’ obesity probability projection algorithm 505) that projects the user's obesity probability. The obesity projection algorithm may be understood as an algorithm that calculates a probability of occurrence of obesity within a predetermined period of time based on a user's health metric (particularly, a health metric for the user's health history characteristics and perceived health characteristics). The data processing module 500 may calculate an obesity probability projection of the user according to an obesity projection algorithm.

Meanwhile, the data extracting module 600 may generate results and content for providing a user-customized obesity solution based on information derived based on a plurality of algorithms 501 to 505. The data extracting module 600 may generate content including results of obesity type to be provided to the user, based on the latent obesity type of the user derived according to the obesity type algorithm (101 obesity type metric algorithm 503). In addition, the data extracting module 600 may derive an obesity inducing marker or an Illness & Obesity Inducing Stress Marker (IOI SM) score 603 based on the ‘101 ObTI vs. Environment & Lifestyle’ compatibility algorithm 504. Also, the content extracting module 600 may project 602 the user's illness and obesity occurrence probability based on the scoring algorithm 502. In addition, in the present disclosure, based on the information derived by the data extracting module 600, a user-customized 5-step obesity solution 604 may be generated.

Meanwhile, the priming module 300, the data collecting module 400, the data processing module 500, and the data extracting module 600 may be understood as one component of the processor 202. The processor 202 may perform data processing for functions of the priming module 300, the data collecting module 400, the data processing module 500, and the data extracting module 600 using the data stored in the memory 206. Hereinafter, the priming module 300, the data collecting module 400, the data processing module 500, and the data extracting module 600 will be referred to as the processor 202 and described without being separately distinguished.

### 2. Orientation Assessment

In the present disclosure, a process of providing a user with an orientation assessment questionnaire configured to measure a plurality of independent orientation parameters including perception orientation, conception orientation, and behavioral orientation (see S410 in FIG. 4).

As described above, the processor 202 may perform a user priming process before performing the user orientation assessment. When the processor 202 determines that the user is ready to start the orientation assessment, the processor 202 may proceed with the orientation assessment by referring to the memory 206. For example, as shown in (a) in FIG. 5, the processor 202 may first provide guide information such as description information, guideline information, and required time information for orientation assessment. In addition, as shown in (b) in FIG. 5, the process may provide an orientation assessment questionnaire to the user terminal 10.

The cognitive behavioral orientation questionnaire may include a questionnaire item (or a questionnaire item and an answer item) associated to each of a plurality of preset orientation parameters in order to assess the user's orientation.

As shown in FIG. 7, in the present disclosure, a plurality of orientation parameters may include at least one of i) perception orientation 710, ii) conception Orientation 720, iii) physical aptness orientation 730, iv) self-directedness tendency 740, v) social interaction tendency 750, and vi) task processing tendency 760.

Meanwhile, in the present disclosure, a group including at least one orientation parameter may be referred to as a “domain (or region)”. A group including the parameters of perception orientation 710, conception orientation 720, and physical aptness orientation 730 may be named “cognitive orientation” or “physical aptness domain”. In addition, a group including parameters of self-directedness tendency 740, social interaction tendency 750, and task processing tendency 760 is named “behavioral tendencies” or “preferences domain”. In addition, in the present disclosure, domains and parameters may be used interchangeably. For example, the behavioral tendencies domain may also be named and described as a behavioral tendencies parameter.

Meanwhile, questionnaire data matched to each of a plurality of independent orientation parameters may exist in the memory 206. For example, it is assumed that “am not interested in other people's problems (first questionnaire data)”, “take time out for other (second questionnaire data)”, “people feel at ease (third questionnaire data)” exist in the memory 206. The first questionnaire data is matched with a first orientation parameter (e.g., perceptual orientation parameter 710) among the plurality of orientation parameters 710 to 760, the second questionnaire data is matched with a second orientation parameter (e.g., conception orientation 720) among the plurality of orientation parameters 710 to 760, and a third orientation data may be matched with a third orientation parameter (e.g., physical aptness orientation 730) among the plurality of questionnaire orientation parameters 710 to 760. Meanwhile, the above-described matching relationship between questionnaire data and orientation parameters is only an example for explanation, and the matching relationship may be variously determined by the manager of the processor 202 or computing device 200 in consideration of the relationship between questionnaire data and orientation parameters.

The processor 202 may provide a questionnaire including a plurality of questionnaire items for orientation assessment to the user terminal 10 based on questionnaire data existing in the memory 206.

### 3. Obesity Assessment

In the present disclosure, a process of providing a user with an obesity assessment questionnaire configured to measure health characteristics including a personal health history, current health, and awareness of personal health may be performed (see S420 in FIG. 4).

As described above, in the present disclosure, a latent obesity type of the user and an obesity probability projection of the user may be determined by combining the results of the cognitive behavioral orientation assessment and obesity assessment of the user. As for the assessment order, the obesity assessment may be performed after the cognitive behavioral orientation assessment is performed, or the cognitive behavioral orientation assessment may be performed after the obesity assessment is performed. Alternatively, it is also possible that the cognitive behavioral orientation assessment and the obesity assessment are performed simultaneously. However, in the present disclosure, for convenience of description, it is described that cognitive behavioral orientation assessment is performed and then, obesity assessment is performed, but it is obvious that the order may be configured in various ways.

As shown in (a) in FIG. 6, the processor 202 may first provide guide information such as description information, guideline information, and required time information for obesity assessment. In addition, as shown in (b) in FIG. 6, the processor 202 may provide the user terminal 10 with a questionnaire for assessing cognitive behavioral orientation.

The obesity assessment questionnaire may include questionnaire items (or questionnaire items and response items) associated to each of a plurality of preset health characteristics (which may also be referred to as health orientation parameters).

As shown in FIG. 9, in the present disclosure, a plurality of health characteristics may include at least one of the user's current or past i) physical environment 910, ii) lifestyle 920, iii) physical condition 930, iv) personal health history 940, v) personal health awareness 950.

Questionnaire data matched to each of a plurality of health characteristics may exist in the memory 206. For example, questionnaire data such as “physical movement/labor” and “communication with other via phone in-person” exist in the memory 206, and such questionnaire data may be matched to a first health characteristic (e.g., environment, 910) among a plurality of health characteristics 910 to 950. For another example, questionnaire data such as “being at the office” and “being mostly at home” exist in the memory 206, and such questionnaire data may be matched to a second health characteristic (e.g., lifestyle 920) among the plurality of health characteristics 910 to 950.

The processor 202 may provide a questionnaire including a plurality of questionnaire items for assessing obesity to the user terminal 10 based on the questionnaire data existing in the memory 206.

Meanwhile, as shown in FIG. 9, at least one health assessment item (which can also be named “sub health characteristic” or “lower health characteristic”) may be matched to present in each of the plurality of health characteristics 910 to 950.

The health assessment item matched to a first health characteristic (e.g., Environment 910) may include at least one of current profession field of study or past career, work type and amount shift schedule, family relationship & personal relationship, and financial stability & financial leeway.

The health assessment item matched to a second health characteristic (e.g., lifestyle, 920) may include at least one of type of food & amount of food, eating pattern & frequency special diet), alcohol consumption style, drinking amount & frequency, sleep duration and time sleep quality and problem, exercise style and exercise frequency and duration, type of personal activities, frequency & skill Level, and socializing style & frequency and communication style.

The health assessment item matched to a third health characteristic (e.g., physical condition 930) may include at least one of genetic health issues & early age health issues, mental health issues and medication and therapy, bowel movement, allergic and auto-immune Issues, weight, height & body shape.

The health assessment item matched to a fourth health characteristic (e.g., personal health history 940) may include at least one of early age body shape and body shapes of parents, 3 significant life events, their Types & when those events happened, past physical health issues & past mental health issues, past drug use & past alcohol use.

The health assessment item matched to a fifth health characteristic (e.g., awareness of personal health 950) may include at least one of easily get fat/lose weight control weight-exercise/diet, comfy/ashamed of own body & confident about own health, work & life balance, anxiety & depression level.

The questionnaire data matched to a specific health characteristic may be matched with at least one of a plurality of health assessment items included in the specific health characteristic. The processor 202 may compute (calculate) health metrics for health characteristics or health assessment items matched with the questionnaire data, based on the user responses to the questionnaire data.

For example, suppose that specific questionnaire data is matched to the first health characteristic (e.g., environment 910). In this case, the specific questionnaire data may be matched with a health assessment item A (current profession field of study or past career) and a health assessment item B (work and amount shift schedule) included in the first health characteristics. The processor 202 may calculate a health metric for the first health characteristic based on a user response to specific questionnaire data. Also, the processor 202 may calculate health metric for each of the health assessment item A and the health assessment item B based on the user's response to the specific questionnaire data.

### 4. Reception of User Response

Meanwhile, in the present disclosure, a process of receiving a user input as a response to an orientation assessment questionnaire and an obesity assessment questionnaire may proceed (see S430 in FIG. 4).

The processor 202 may configure a graphic interface including the questionnaire in various ways in order to receive a user response to the questionnaire.

For example, as shown in (b) in FIG. 5, the processor 202 may display a graphic object corresponding to each of a plurality of answer items (3 to 10 points) on the user terminal 10. The processor 202 may receive a specific answer item as a user response to a specific questionnaire item, based on a user input that moves the location of a graphic object including a specific questionnaire item (e.g., “am not interested in other people's problems”) to a graphic object corresponding to a specific answer item (e.g., 10 points (Most)).

For another example, as shown in (b) in FIG. 6, the processor 202 may display a check box matched with each of a plurality of questionnaire items (or answer items) on the user terminal 10. The processor 202 may receive selection of a questionnaire item (or answer a question) matched with a specific check box as a user response, based on a user input for a specific check box.

### 5. Computation of Orientation Metric

Meanwhile, in the present disclosure, a process of computing an orientation metric corresponding to each independent orientation parameter may proceed according to a user input as a response to an orientation assessment questionnaire (see S440 in FIG. 4).

The processor 202 may compute an orientation metric (which may also be named “score”) for a specific orientation parameter based on user responses to questionnaire items matched with the specific orientation parameter. In this case, the processor may compute the orientation metric for each orientation parameter based on a predefined cognitive behavioral orientation algorithm (or directedness algorithm, see reference numeral 501 in FIG. 3) to compute the orientation metric of each orientation parameter.

For each of a plurality of orientation parameters, reference information that is a basis for determining a level corresponding to the orientation metric may be matched and present in the memory 206. The processor 202 may determine a level for each orientation parameter based on the reference information.

As shown in FIG. 7, the reference information matched to the first orientation parameter (perception orientation 710) may include a first level (perceiving the entire scene including an Object) corresponding to the orientation metric (e.g., 1 to 5 points) of a first domain, a second level (perceiving the relationship of an Object & its surrounding) corresponding to the orientation metric (e.g., 6 to 10 points) of a second domain, a third level (perceiving the entirety of an Object) corresponding to the orientation metric (e.g., 11 to 15 points) of a third domain, and a fourth level (perceiving zoom-in, partial details of an Object) corresponding to the orientation metric (16 to 20 points) of a fourth domain.

The processor 202 may calculate the orientation metric (e.g., “12 points”) corresponding to the first orientation parameter based on a user response to a question item matched to the first orientation parameter. Further, the processor 202 may determine the level (perceiving the entirety of an Object) corresponding to the orientation metric (e.g., “12 points”) computed based on the reference information matched with the first orientation parameter, as the user level associated with the first orientation metric.

Furthermore, the reference information matched to the second orientation parameter (conception Orientation 720) may include a first level (convergently deducing though) corresponding to the orientation metric (e.g., 1 to 5 points) of a first domain, a second level (causal relationship oriented thoughts) corresponding to the orientation metric (e.g., 6 to 10 points) of a second domain, a third level (alternative solutions oriented thoughts) corresponding to the orientation metric (e.g., 11 to 15 points) of a third domain, and a fourth level (divergently diffusing thoughts) corresponding to the orientation metric (e.g., 16 to 20 points) of a fourth domain.

The processor 202 may compute an orientation metric (e.g., “6 points”) corresponding to the second orientation parameter based on a user response to a questionnaire item matched to the second orientation parameter. Then, the processor 202 may determine the level (causal relationship oriented thoughts) corresponding to the orientation metric (e.g., “6 points”) computed based on the reference information matched with the second orientation parameter, as the user level associated with the second orientation metric.

Furthermore, the reference information matched to the third orientation parameter (physical aptness orientation 730) may include a first level (physically sedentary) corresponding to the orientation metric (e.g., 1 to 5 points) of a first domain, a second level (activity selective) corresponding to the orientation metric (e.g., 6 to 10 points) of a second domain, a third level (experience oriented) corresponding to the orientation metric (e.g., 11 to 15 points) of a third domain, and a fourth level (dynamic & relentless) corresponding to the orientation metric (e.g., 16 to 20 points) of a fourth domain.

The processor 202 may compute an orientation metric (e.g., “11 points”) corresponding to the third orientation parameter based on a user response to a questionnaire item matched to the third orientation parameter. Then, the processor 202 may determine the level (experience oriented) corresponding to the orientation metric (e.g., “11 points”) computed based on the reference information matched with the third orientation parameter, as the user level associated with the third orientation metric.

Furthermore, the reference information matched to the fourth orientation parameter (self-directedness tendency 740) may include a first level (driven to maintain) corresponding to the orientation metric (e.g., 1 to 5 points) of a first domain, a second level (driven to mediate) corresponding to the orientation metric (e.g., 6 to 10 points) of a second domain, a third level (driven to coordinate) corresponding to the orientation metric (e.g., 11 to 15 points) of a third domain, and a fourth level (driven to lead) corresponding to the orientation metric (e.g., 16 to 20 points) of a fourth domain.

The processor 202 may compute an orientation metric (e.g., “16 points”) corresponding to the fourth orientation parameter based on a user response to a questionnaire item matched to the fourth orientation parameter. Then, the processor 202 may determine the level (driven to lead) corresponding to the orientation metric (e.g., “16 points”) computed based on the reference information matched with the fourth orientation parameter, as the user level associated with the fourth orientation metric.

Furthermore, the reference information matched to the fifth orientation parameter (social interaction tendency 750) may include a first level (relationship limited) corresponding to the orientation metric (e.g., 1 to 5 points) of a first domain, a second level (relationship selective) corresponding to the orientation metric (e.g., 6 to 10 points) of a second domain, a third level (embracing all members in my network) corresponding to the orientation metric (e.g., 11 to 15 points) of a third domain, and a fourth level (embracing all people) corresponding to the orientation metric (e.g., 16 to 20 points) of a fourth domain.

The processor 202 may compute an orientation metric (e.g., “14 points”) corresponding to the fifth orientation parameter based on a user response to a questionnaire item matched to the fifth orientation parameter. Then, the processor 202 may determine the level (embracing all members in my network) corresponding to the orientation metric (e.g., “14 points”) computed based on the reference information matched with the fifth orientation parameter, as the user level associated with the fifth orientation metric.

Furthermore, the reference information matched to the sixth orientation parameter (task processing tendency 760) may include a first level (driven to preference and ability to perform) corresponding to the orientation metric (e.g., 1 to 5 points) of a first domain, a second level (driven to prioritizing and delegating responsibilities) corresponding to the orientation metric (e.g., 6 to 10 points) of a second domain, a third level (driven to completing all responsibilities) corresponding to the orientation metric (e.g., 11 to 15 points) of a third domain, and a fourth level (processing beyond current responsibilities) corresponding to the orientation metric (e.g., 16 to 20 points) of a fourth domain.

The processor 202 may compute an orientation metric (e.g., “9 points”) corresponding to the sixth orientation parameter based on a user response to a questionnaire item matched to the sixth orientation parameter. Then, the processor 202 may determine the level (driven to prioritizing and delegating responsibilities) corresponding to the orientation metric (e.g., “9 points”) computed based on the reference information matched with the sixth orientation parameter, as the user level associated with the sixth orientation metric.

### 6. Computation of Health Metric

Meanwhile, in the present disclosure, a process of computing a health metric corresponding to each of a plurality of health characteristics may proceed according to a user input as a response to an obesity assessment questionnaire (see S450 in FIG. 4).

The processor 202 may calculate a health metric for a specific health characteristic based on user responses to questionnaire items matched to the specific health characteristic. In addition, the processor 202 may calculate a health metric for a specific health assessment item based on user responses to questionnaire items matched to a specific health assessment item (“sub health characteristic” or “lower health characteristic”).

In this case, the processor 202 uses a predefined algorithm for each of a plurality of health characteristics (scoring algorithm for each category of five areas, see reference numeral 502 in FIG. 3) to compute (calculate, obtain) a health metric (also named “score”) for each health characteristic.

For example, the processor 202 may compute a health metric corresponding to a first health characteristic based on user responses to questionnaire items matched to the first health characteristic (e.g., physical environment 910). In this case, the processor 202 may compute a health metric (e.g., “85 points”) for the first health characteristic using a scoring algorithm predefined for the first health characteristic. In addition, the processor 202 may compute a health metric for each of the health assessment items (“sub health characteristic” or “lower health characteristic”) included in the first health characteristics, based on user responses to questionnaire items matched with the first health characteristic.

In another example, the processor 202 may compute a health metric corresponding to a second health characteristic based on user responses to questionnaire items matched to the second health characteristic (e.g., lifestyle 920). In this case, the processor 202 may compute a health metric (e.g., “35 points”) for the second health characteristic using a predefined scoring algorithm for the second health characteristic. In addition, the processor 202 may compute a health metric for each of the health assessment items (“sub health characteristic” or “lower health characteristic”) included in the second health characteristics, based on user responses to questionnaire items matched with the second health characteristic.

### 7. Determination of Latent Obesity Type+Obesity Probability Projection

Meanwhile, in the present disclosure, a process of determining a latent obesity type for a user and an obesity probability projection for the user may proceed according to the computed orientation metric and the computed health metric (see S460 in FIG. 4).

The processor 202 may determine a latent obesity type for the user by using a 101 obesity type metric algorithm (see reference numeral 503 in FIG. 3). In addition, the processor 202 may determine an obesity probability projection for the user by using a plurality of different algorithms (‘101 ObTI vs. Environment & Lifestyle’ compatibility algorithm and ‘+History & Awareness’ obesity probability prediction algorithm, see reference numerals 504 and 505 in FIG. 3). Hereinafter, the process of determining the latent obesity type and obesity probability projection for the user will be described in turn.

### 7-1. Determination of Latent Obesity Type

The processor 202 may determine a latent obesity type of the user based on at least some of the orientation metric and the health metric. The processor 202 may use a predefined obesity type algorithm (or 101 obesity type metric algorithm) to determine a latent obesity type.

As shown in FIG. 8A, the obesity type algorithm may classify the user as one of a plurality of obesity groups (A-1a, A-1b, A-2a, A-2b, B-1a, B-1b, B-2a, and B-2b) based on a combination of at least some of the orientation metric and the health metric. The obesity type algorithm may use an orientation metric corresponding to a cognitive domain in first and second classification processes, and may use an orientation metric corresponding to a behavioral domain in a third classification process.

Specifically, the processor 202 may specify a group to which the user belongs among group A (Low physical engagement) and group B (High physical engagement), based on the orientation metric corresponding to a first specific parameter (at least some of the orientation metrics corresponding to the cognitive domain, for example, it may be an orientation metric corresponding to a “physical aptness orientation”). If the orientation metric corresponding to the first specific parameter is less than a certain reference metric (e.g., 11 points), the processor 202 may specify the group corresponding to the user as group A, and if the orientation metric corresponding to the physical aptness orientation parameter is equal to or greater than a certain reference metric (e.g., 11 points), the processor 202 may specify the group corresponding to the user as group B.

Secondarily, the processor 202 may specify a group to which the user belongs among a A-1 group (LOW physical engagement and LOW cognitive engagement), a A-2 group (LOW physical engagement but HIGH cognitive engagement), a B-1 group (HIGH physical engagement but LOW cognitive engagement), and a B-2 group (HIGH physical engagement and HIGH cognitive engagement), based on the orientation metric for a second specific orientation parameter (at least some of the orientation metrics corresponding to the cognitive domain, for example, an orientation metric corresponding to at least one parameter of “perception orientation and conception orientation). In this case, the processor 202 may specify the group corresponding to the user as the A-1 group or the B-1 group if the orientation metric corresponding to the cognitive orientation parameter is less than a certain reference metric (e.g., 11 points), and the processor 202 may specify the group corresponding to the user as the A-2 group or the B-2 group if the orientation metric corresponding to the cognitive orientation parameter is equal to or greater than a certain reference metric (e.g., 11 points).

Thirdly, the processor 202 may specify one of eight obesity groups as a group to which the user belongs, based on the orientation metric corresponding to the third specific orientation parameter (which may correspond to at least some of the orientation metrics included in the behavioral domain).

The eight obesity groups may include i) a first obesity group (A-1a group: combination of Low physical engagement+Low cognitive engagement+Negative behavioral engagement), ii) a second obesity Group (A-1b group: combination of Low physical engagement+Low cognitive engagement+Positive behavioral engagement), iii) a third obesity group (A-2a group: combination of Low physical engagement+High cognitive engagement+Negative behavioral engagement), iv) a fourth obesity group (A-2b Group: combination of Low physical engagement+High cognitive engagement+Positive behavioral engagement), v) a fifth obesity group (Group B-1a: combination of High physical engagement +Low cognitive engagement+Negative behavioral engagement), vi) a sixth obesity group (B-1b group: combination of High physical engagement+Low cognitive engagement+Positive behavior engagement), vii) a seventh obesity group (Group B-2a: combination of High physical engagement+High cognitive engagement+Negative behavioral engagement), and viii) an eighth obesity group (B-2b group: combination of High physical engagement+High cognitive engagement+Positive behavioral engagement).

For example, it is assumed that the orientation metric corresponding to the first specific orientation parameter for the user is “8 points”, the orientation metric corresponding to the second specific orientation parameter is “3 points”, and the orientation metric corresponding to the third specific orientation parameter is “10 points”. The processor 202 may firstly classify the user into group A, secondly classify the user into group A-1, and thirdly classify the user into group A-1a according to the obesity type algorithm.

On the other hand, as shown in FIG. 8b, in each of the plurality of obesity groups (A-1a, A-1b, A-2a, A-2b, B-1a, B-1b, B-2a, B-2b), the obesity types may be matched and present. These types of obesity may belong to a category representing a specific body engagement level, a specific cognitive engagement level, and a specific behavioral engagement level derived from the computed orientation metrics. Specifically, the obesity types may be clustered into 101 types corresponding to the obesity groups among all patterns (e.g., “8,192 patterns”) which can be obtained by combining the orientation metrics corresponding to the plurality of orientation parameters. Obesity type may be the most common pattern seen in the respective obesity groups. Each of the obesity types can consist of a 5-digit code. The first 3 digits of the 5-digit code may correspond to an orientation metric derived from the cognitive domain, and the remaining 2 digits may correspond to an orientation metric derived from the behavioral domain. For example, the first 3 digits “111” of the obesity type “111AA(801)” matched to the A-1a group may correspond to the orientation metric derived from the cognitive domain, and the remaining 2 digits “AA” may correspond to the orientation metric derived from the behavioral domain.

The processor 202 may specify, as the latent obesity type of the user, one of the obesity types matched to the obesity group to which the user belongs, based on the orientation metric for the user. For example, it is assumed that a user is matched to group A-1a. The processor 202 may specify a latent obesity type for the user as “111AM 802” based on the user's orientation metric.

In this way, the processor 202 may specify the latent obesity type of the user from different options according to which of the plurality of obesity groups the user belongs to. Furthermore, the processor 202 may specify latent obesity types of the respective users differently based on different orientation metric results of the users even though the users belong to the same obesity group.

### 7-2. Determination of Obesity Inducing Stress Marker

Meanwhile, the processor 202 may determine one or more obesity inducing stress markers for the user according to the computed orientation metric and a first set of computed health metrics corresponding to the user's current physical environment and current lifestyle.

The processor 202 may generate a new orientation metric based on some of the orientation metrics and health metrics (e.g., a health metric corresponding to the physical aptness orientation 730). The processor 202 may compute an orientation metric for each of a plurality of predefined orientation items based on the user's latent obesity type and some of the user's health metrics.

For example, as shown in FIG. 10, the processor 202 may compute the orientation metric for each of the 10 orientation items. In this case, the orientation items may include at least one of i) compatible career, ii) compatible task environment, iii) relationship sensitivity, iv) financial susceptibility, and v) compatible diet style), vi) compatible sleep style, vii) compatible exercise style, viii) compatible hobby, ix) compatible socializing style, x) self-disciplinary style.

Hereinafter, the existing orientation metric computed from the response to the orientation questionnaire and the new orientation metric newly constructed based on the orientation metric will be named as orientation metrics without distinction and described.

Meanwhile, the processor 202 may determine the obesity inducing markers that cause obesity of the user based on the orientation metric and the health metric.

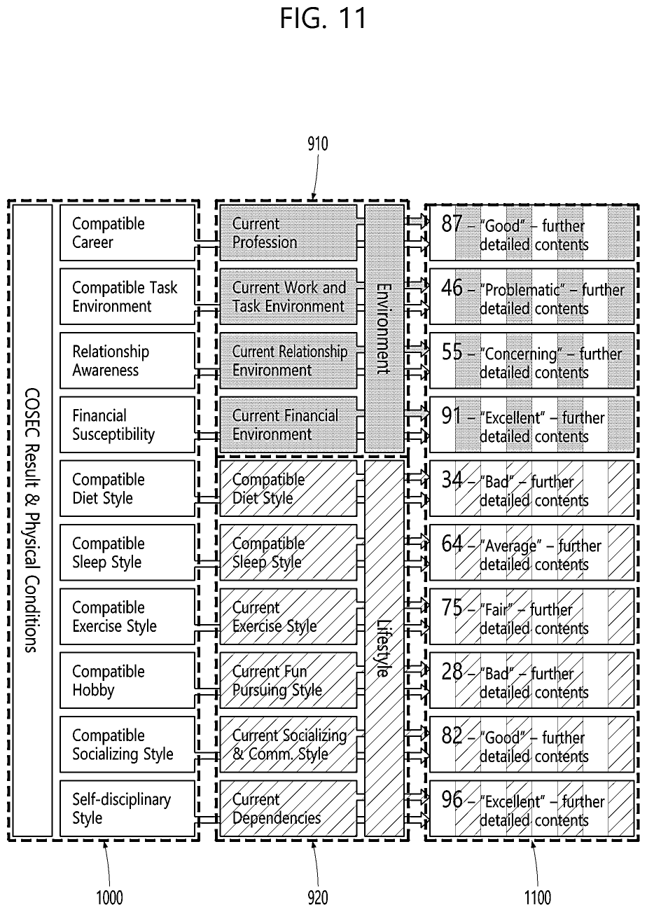

Specifically, as shown in FIG. 11, the processor 202 may use health metrics corresponding to specific health characteristics (physical environment characteristics and lifestyle characteristics, 910 and 920) among a plurality of health characteristics to construct a first set of health metrics. The processor 202 may compute a compatibility metric (or compatibility score) by comparing items compatible (corresponding or matching) between the orientation metrics 1000 and the first set of health metrics. In addition, the processor 202 may determine an obesity inducing marker based on a specific item if the compatibility metric of the specific item is less than a preset value (e.g., 50 points). For example, it is assumed that the compatibility metric of the specific item “compatible diet style” in the orientation metrics 1000 and the corresponding item “current diet style” in the first set of health metrics is computed as “34”. The processor 202 may determine a “diet” associated with a specific item as an obesity inducing marker.

Meanwhile, the processor 202 may compute a compatibility metric by comparing matching items between the orientation metrics 1000 and the first set of health metrics.

In this case, items matching each other may be understood as items having similar or corresponding meanings. For example, the processor 202 may compute a compatibility metric by comparing the respective items based on a correspondence between the meanings of a first item (e.g., “compatible career”) meaning a suitable career in the orientation metrics 1000 and a first item (e.g., “current profession”) meaning a current career in a first health characteristic 910. As another example, the processor 202 may compute a compatibility metric by comparing the respective items based on a correspondence between the meanings of a second item (e.g., “compatible task environment”) meaning a suitable task environment in the orientation metrics 1000 and a second item (e.g., “current work and task environment”) meaning the current task and task environment in the first health characteristic 910.

The processor 202 may determine at least one health inducing stress marker 1100 based on the health metric matched to the orientation metrics 1000, the physical environment characteristic 910, and the lifestyle characteristic 920.

In this case, the obesity inducing stress marker 1100 may include stress grades indicating excellent, good, fair, average, bad, concerning, problematic, and the like, based on the computed compatibility score.

For example, as shown in FIG. 11, the processor 202 may assign a corresponding grade if the computed score is a score corresponding to “excellent” (e.g., 90 or higher), and may assign a corresponding grade if the computed score corresponds to “bad” (e.g., 30 or less). In addition, the processor 202 may include the assigned assessment grade in the obesity inducing stress marker 1100.

### 7-3. Determination of Obesity Incidence Precursor

Meanwhile, the processor 202 may determine an obesity incidence precursor projection for computing an obesity probability projection for the user.

The processor 202 may determine the obesity incidence precursor projection for the user according to the obesity inducing stress marker 1100 determined for the user and a second set of computed health metric corresponding to personal health history and awareness of personal health.

In this case, the second set may include a health metric corresponding to the personal health history characteristic 930 and a health metric corresponding to the personal health awareness characteristic 950 among a plurality of health characteristics.

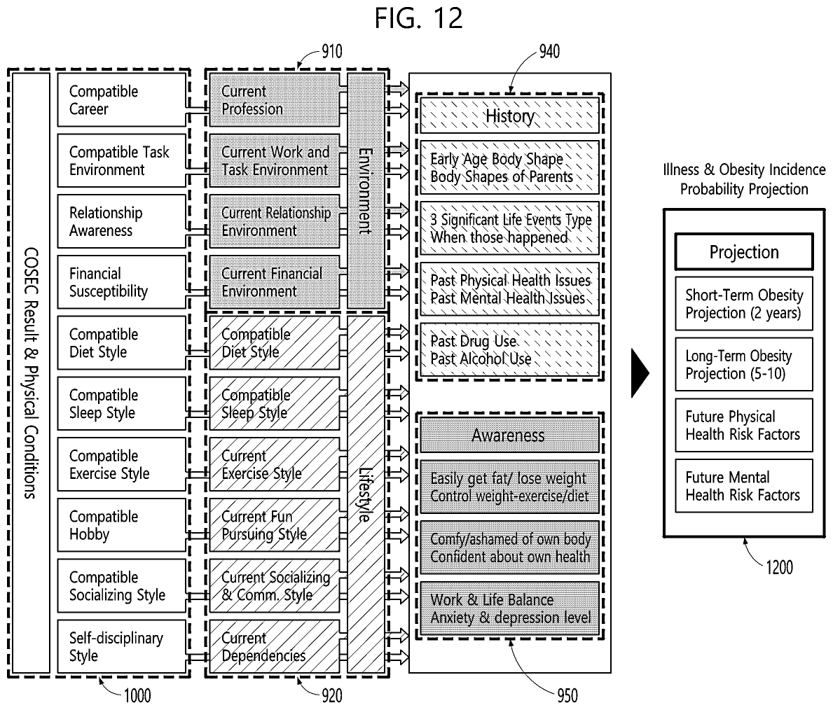

As shown in FIG. 12, the processor 202 may determine the obesity inducing stress marker based on the orientation metrics 1000 and the first set of health metrics (consisting of health metrics for physical environment characteristic 910 and health metrics for lifestyle 920), and may determine an obesity incidence precursor projection 1200 based on the second set of health metrics (consisting of health metrics for personal health history characteristic 940 and personal health awareness characteristic 950) therein.

In this case, the obesity incidence precursor projection 1200 may include a plurality of items including information about the user's obesity probability projection.

A first item (e.g., “short-term obesity projection (2 years)”) of the plurality of items of obesity incidence precursor projections 1200 may mean “short-term obesity projection (2 years)”. Specifically, it may mean projecting changes in weight and body composition of an individual over a period of two years, analyzing factors related to obesity, and performing projection in consideration of an individual's health condition, eating habits, exercise level, and the like.

A second item (e.g., “long-term obesity projection (5-10)”) means “long-term obesity projection (5 to 10 years). Specifically, it may mean projecting changes in weight and body composition of an individual over a period of 5 to 10 years, analyzing factors related to obesity, and performing projection in consideration of an individual's health condition, eating habits, exercise levels, and the like.

A third item (e.g., “future physical health risk factors”) means “future physical health risk factors”. Specifically, it may mean factors that are likely to cause an individual to physically suffer from a disease or health problem in the future, and these risk factors may be caused by various factors such as the individual's health condition, genetic factors, lifestyle, environmental factors, and the like.

A fourth item (e.g., “future mental health risk factors”) means “future mental health risk factors”. Specifically, it may mean factors that make an individual highly likely to suffer from mental illness or mental health problems in the future, and the mental health risk factors may be caused by various factors such as the individual's current mental state, genetic factors, physical environment, stress factors, and the like.

The processor 202 may determine an obesity probability projection for the user based on the obesity incidence precursors. In this way, the obesity probability projection determined by the obesity incidence precursor projection 1200 may represent the probability of obesity occurring within a predetermined time (e.g., unspecified future time point, 2 years, 5 years, 10 years, etc.). Accordingly, the obesity probability projection for the user may be determined based on the obesity incidence precursor projection 1200.

### 8. Generation and Provision of an Obesity Mitigation Action Plan

Meanwhile, in the present disclosure, a process of generating an obesity mitigation action plan with a plurality of steps in accordance with the determined latent obesity type and the obesity probability projection may be proceeded (see S470 in FIG. 4).

An obesity mitigation action plan may refer to specific plans and goals set by individuals to alleviate obesity and maintain a healthy weight. For example, it may be understood as setting a goal to obtain an effect for preventing or alleviating obesity, including an individual's diet, exercise, and lifestyle.

The processor 202 may utilize the obesity inducing stress markers 1100 determined for the user to generate an obesity mitigation action plan.

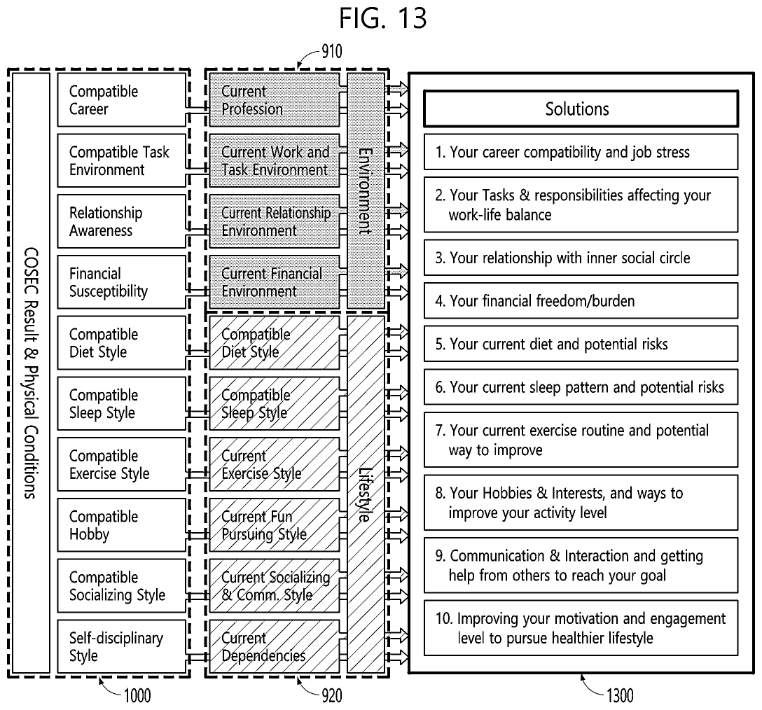

More specifically, the processor 202 may generate a solution set 1300 for generating an obesity mitigation action plan (see FIG. 13). The solution set 1300 may be generated based on the orientation metric 1000, the first set of health metrics (health metrics corresponding to physical environment characteristic 910 and lifestyle characteristic 920). In this case, the solution set 1300 may include a plurality of solution items corresponding to items matched between the orientation metrics 1000 and the first set of health metrics.

Hereinafter, a plurality of solution items included in the solution set 1300 will be described in detail.

The processor 202 may generate a first solution item (e.g., “your career compatibility and job stress”) corresponding to the first item (e.g., “compatible career”) of the orientation metrics 1000 and the first item (e.g., “current profession”) of the first set of health metrics. In this case, the first solution item may mean “your career compatibility and job stress”.

The processor 202 may generate a second solution item (e.g., “your tasks & responsibilities affecting your work-life balance”) corresponding to the second item (e.g., “compatible task environment”) of the orientation metrics 1000 and the second item (e.g., “current work and task environment”) of the first set of health metrics. In this case, the second solution item may mean “How your job responsibilities affect your work-life balance”.

The processor 202 may generate a third solution item (e.g., “your relationship with inner social circle”) corresponding to the third item (e.g., “relationship awareness”) of the orientation metrics 1000 and the third item (e.g., “relationship environment”) of the first set of health metrics. In this case, the third solution item may mean “your social relationships (or reciprocal relationships)”.

The processor 202 may generate a fourth solution item (e.g., “your financial freedom/burden”) corresponding to the fourth item (e.g., “financial susceptibility”) of the orientation metrics 1000 and the fourth item (e.g., “current financial environment”) of the first set of health metrics. In this case, the fourth solution item may mean “your financial freedom/burden”.

The processor 202 may generate a fifth solution item (e.g., “your current diet and potential risks”) corresponding to the fifth item (e.g., “compatible diet style”) of the orientation metrics 1000 and the fifth item (e.g., “current diet style”) of the first set of health metrics. In this case, the fifth solution item may mean “your current diet and potential risks”.

The processor 202 may generate a sixth solution item (e.g., “your current sleep pattern and potential risks”) corresponding to the sixth item (e.g., “compatible sleep style”) of the orientation metrics 1000 and the sixth item (e.g., “current sleeping style”) of the first set of health metrics. In this case, the sixth solution item may mean “your current sleep pattern and potential risks”.

The processor 202 may generate a seventh solution item (e.g., “your current exercise routine and potential way to improve”) corresponding to the seventh item (e.g., “compatible exercise style”) of the orientation metrics 1000 and the seventh item (e.g., “current exercise style”) of the first set of health metrics. In this case, the seventh solution item may mean “your current exercise routine and potential way to improve”.

The processor 202 may generate an eighth solution item (e.g., “your hobbies & interests, and ways to improve your activity level”) corresponding to the eighth item (e.g., “compatible hobby”) of the orientation metrics 1000 and the eighth item (e.g., “current fun pursuing style”) of the first set of health metrics. In this case, the eighth solution item may mean “how to increase your activity level for your hobbies and interests”.

The processor 202 may generate a ninth solution item (e.g., “communication & interaction and getting help from others to reach your goal”) corresponding to the ninth item (e.g., “compatible socializing style”) of the orientation metrics 1000 and the ninth item (e.g., “current socializing & communication style”) of the first set of health metrics. In this case, the ninth solution item may mean “Get help from others for communicate and interact and achieving goals”.

The processor 202 may generate a tenth solution item (e.g., “improving your motivation and engagement level to pursue healthier lifestyle”) corresponding to the tenth item (e.g., “self-disciplinary style”) of the orientation metrics 1000 and the tenth item (e.g., “current dependencies”) of the first set of health metrics. In this case, the tenth solution item may mean “increased motivation and engagement levels to pursue healthier living”.

Meanwhile, the processor 202 may generate an obesity mitigation action plan consisting of a plurality of steps based on the solution set 1300.

The processor 202 may generate an obesity mitigation action plan consisting of a plurality of steps (or a plurality of procedures or a plurality of processes). Among the plurality of procedures, a first procedure may relate to problem awareness, a second procedure may relate to problem recognition, a third procedure may relate to determination, a fourth procedure may relate to planning, and a fifth procedure may relate to execution & feedback Loop. That is, the processor 202 may generate an obesity mitigation action plan consisting of a total of 5 steps (or 5 procedures). Accordingly, in the present disclosure, it may also be referred to as “5 Stages of solution process”.

A problem awareness step 1410 in the obesity mitigation action plan is the first step in the obesity mitigation action plan, and means the stage of being aware of the existence of a problem. At this stage, it is important to aware the problems that arise in the surrounding environment or situation, and to understand the impact of those problems on oneself or the surroundings. Accordingly, the problem awareness step 1410 may include a plurality of items related to the living environment and lifestyle included in the obesity inducing stress marker 1100. It can also be understood as being associated with a plurality of items included in the solution set 1300.

For example, a plurality of items included in the problem awareness step 1410 (e.g., awareness of the existence of a problem) may include job, tasks and responsibilities, friends and family, finances, diet/eating habits, sleep, exercise, and interests, communication and interaction, determination/willpower, and the like.

The problem recognition step 1420 is the second step in the obesity mitigation action plan, and means the stage of understanding the seriousness of the problem and its ramifications beyond the stage of recognizing the problem. In the problem recognition step 1420, issues, disagreements, challenges, complaints, etc. generated in a given situation may be recognized and identified as problems.

The determination (or decision) step 1430 is the third step in the obesity mitigation action plan, and means the stage for determining the will to solve the problem. In the determination step 1430, various alternatives for solving the problem may be considered, and the most appropriate solution may be selected among them.

The planning step 1440 is the fourth step in the obesity mitigation action plan, and means the stage for determining a series of actions and procedures necessary to achieve the goal. In the planning step 1440, it is possible to set a goal and plan and organize a plan to proceed accordingly.

The execution and feedback step 1450 is the fifth step in the obesity mitigation action plan, and means the stage of actually executing the plan and collecting assessment and feedback on the result. In the execution and feedback step 1450, implementation and feasibility of the plan may be identified, adjustments may be made as necessary, and continuous improvement may be performed.

On the other hand, at least one of a plurality of steps related to the obesity mitigation action plan may provide exercise instructions to be followed by the user.

For example, the processor 202 may provide exercise instructions for the user to follow through planning step 1440 of the plurality of steps. In this case, exercise instructions may be provided customized according to the user's individual degree of obesity (severe obesity, overweight, general obesity, and reduced muscle mass obesity).

In addition, at least one of the plurality of steps related to the obesity mitigation action plan may receive user feedback in response to the action plan.

In this case, receiving the user's feedback may be understood as receiving opinions and assessments from the user for at least one plan among a plurality of obesity mitigation action plans.

In this way, the processor 202 may receive the user's feedback request according to the feedback request related to the user's obesity mitigation action plan.

On the other hand, in the present disclosure, a process of displaying the obesity mitigation action plan on a display may proceed (see S470 in FIG. 4).

Specifically, the processor 202 may display the obesity mitigation action plan on a display provided in the user terminal 10.

First, as shown in (a) in FIG. 15, a plurality of items according to a user's examination result may be output as graphic objects on a screen output to a display provided in the user terminal 10.

In addition, the processor 202 may provide different pages based on the graphic object corresponding to each item being selected from the user terminal 10.

For example, a first item (e.g., “obesity type”) may provide a summary page together with an obesity type. A second item (e.g., “obesity type through obti test”) may provide a detailed analysis page of obesity type through an orientation assessment questionnaire. A third item (e.g., “lifestyle & environment) may provide a detailed physical environment and lifestyle analysis page through an obesity assessment questionnaire. A fourth item (e.g., “health projection) may represent an obesity mitigation action plan through an orientation assessment questionnaire and an obesity assessment questionnaire. A fifth item (e.g., “health trend”) may provide a graph showing changes when the orientation assessment questionnaire and the obesity assessment questionnaire are conducted more than twice.

In this case, the plurality of items may be deactivated in a state in which the user does not perform an examination corresponding to each item, and activated when the user performs an examination.

Furthermore, the processor 202 may display an obesity mitigation action plan on the display of the user terminal 10, based on the fourth item (e.g., “health projection) being selected from the user terminal 10.

For example, as shown in (b) in FIG. 15, an obesity mitigation action plan may be displayed on the user terminal 10. The problem awareness step 1410 of the obesity mitigation action plan is the first step in the obesity mitigation action plan, which means awareness of the existence of a problem. At this stage, it is important to be aware of the problems that arise in the surrounding environment or situation, and to understand the impact of those problems on oneself or the surroundings. Accordingly, the problem awareness step 1410 may include a plurality of items related to the physical environment and lifestyle included in the obesity inducing stress marker 1100. It may also be understood as being associated with the plurality of items included in the solution set 1300.

The method for identifying latent obesity according to the present disclosure may provide a user with an orientation assessment questionnaire configured to measure a plurality of independent orientation parameters including perception orientation, conception orientation, and behavior orientation. Through this, the present disclosure may define a user's obesity type and provide a new and improved customized obesity solution based on the user's cognitive and behavioral orientation, unlike the method of specifying the user's obesity type based only on the user's body attributes (age, weight, etc.)

Furthermore, the method for identifying latent obesity according to the present disclosure may provide the user with an obesity assessment questionnaire configured to measure health characteristics including personal health history, current health, and perception of personal health. Through this, the present disclosure may determine the user's obesity type by considering a wide range of factors, including the user's physical condition, lifestyle, and life patterns, beyond the method of determining the user's obesity type based on quantitative body values.

Furthermore, the method for identifying latent obesity according to the present disclosure may compute orientation metrics based on user responses to an orientation assessment questionnaire, and compute health metrics based on user responses to an obesity assessment questionnaire, and determine the latent obesity type and obesity probability projection for the user using the orientation metrics and the health metrics. Through this, the present disclosure may determine the user's obesity type in a systematic and subdivided manner.

Furthermore, the method for identifying latent obesity according to the present disclosure may provide an obesity mitigation action plan consisting of a plurality of steps according to the latent obesity type and the obesity probability projection, thereby providing the user with customized solutions which represent what needs to be improved about future health risks.

Furthermore, the obesity mitigation action plan according to the present disclosure may represent the user's problem awareness, problem severity recognition, and determination (decision) steps to be optimized for each user, which are classified based on a wide range of factors, including the user's plurality of orientation parameters and physical condition, lifestyle, and life patterns, unlike the existing solution method that focuses only on obesity mitigation plan and implementation based only on the user's body attributes (age, weight, etc.), so that the obesity mitigation action plan and execution may be implemented to obtain more effective and realistic results.

Meanwhile, the present disclosure described above may be implemented as a program that is executed by one or more processes in a computer and can be stored in a computer-readable medium (or a recording medium).

Furthermore, the present disclosure described above may be implemented as computer readable codes or instructions in a medium on which a program is recorded. That is, the present disclosure may be provided in the form of a program.

On the other hand, the computer readable medium includes all types of recording devices in which data that can be read by a computer system is stored. Examples of computer readable media include hard disk drive (HDD), solid state disk (SSD), silicon disk drive (SDD), ROM, RAM, CD-ROM, magnetic tape, floppy disk, optical data storage device, etc.

Furthermore, the computer readable medium may be a server or cloud storage that includes storage and can be accessed by electronic devices through communication. In this case, the computer may download the program according to the present disclosure from a server or cloud storage through wired or wireless communication.

Furthermore, in the present disclosure, the above-described computer is an electronic device equipped with a processor, that is, a central processing unit (CPU), and there is no particular limitation on its type.

Meanwhile, the above descriptions are to be considered in all respects as illustrative and not restrictive. The scope of the present disclosure should be determined by reasonable interpretation of the appended claims and all change which comes within the equivalent scope of the disclosure are included in the scope of the disclosure.

What is claimed is:

1. A method for identifying latent obesity, comprising:
at a computing device having a display, one or more processors, and memory storing one or more programs configured for execution by the one or more processors:
providing a user with an orientation assessment questionnaire configured to measure a plurality of independent orientation parameters, including perception orientation, conception orientation, and behavioral orientation;
providing the user with an obesity assessment questionnaire configured to measure health characteristics, including personal health history, current health, and awareness of personal health;
receiving user input in response to the orientation assessment questionnaire and the obesity assessment questionnaire;
for each of the independent orientation parameters, computing a corresponding orientation metric according to the user input in response to the orientation assessment questionnaire;
for each of a plurality of health characteristics, computing a corresponding health metric according to the user input in response to the obesity assessment questionnaire;
in accordance with the computed orientation metrics and the computed health metrics, determining a latent obesity type for the user and an obesity probability projection for the user;
in accordance with the determined latent obesity type and the obesity probability projection, generating an obesity mitigation action plan with a plurality of steps;
displaying the action plan on the display.

2. The method of claim 1, wherein the health characteristics further includes current physical environment and current lifestyle of the user.

3. The method of claim 2, further comprising:
in accordance with the computed orientation metrics and a first set of the computed health metrics corresponding to the current physical environment and current lifestyle of the user, determining one or more obesity inducing stress markers for the user.

4. The method of claim 3, wherein generating the obesity mitigation action plan includes utilizing the determined obesity inducing stress markers for the user.

5. The method of claim 4, further comprising:
in accordance with the determined obesity inducing stress markers and a second set of the computed health metrics corresponding to the personal health history and the awareness of personal health, determining an obesity incidence precursor projection for the user;
wherein the obesity probability projection for the user is further determined based on the determined obesity incidence precursor projection.

6. The method of claim 4, wherein the determined obesity probability projection represents a probability of whether obesity will occur within a predetermined span of time.

7. The method of claim 1, wherein at least one of the plurality of steps includes receiving user feedback in response to the action plan.

8. The method of claim 1, wherein at least one of the plurality of steps includes providing exercise instructions for the user to follow.

9. The method of claim 1, wherein the latent obesity type belongs to a category that represents a particular physical engagement level, a particular cognitive engagement level, and a particular behavioral engagement level derived from the computed orientation metrics.

10. A computing device, comprising:
one or more processors;
memory;
one or more programs stored in the memory and configured for execution by the one or more processors, the one or more programs including instructions for:
providing a user with an orientation assessment questionnaire configured to measure a plurality of independent orientation parameters, including perception orientation, conception orientation, and behavioral orientation;
providing the user with an obesity assessment questionnaire configured to measure health characteristics, including personal health history, current health, and awareness of personal health;
receiving user input in response to the orientation assessment questionnaire and the obesity assessment questionnaire;
for each of the independent orientation parameters, computing a corresponding orientation metric according to the user input in response to the orientation assessment questionnaire;
for each of a plurality of health characteristics, computing a corresponding health metric according to the user input in response to the obesity assessment questionnaire;
in accordance with the computed orientation metrics and the computed health metrics, determining a latent obesity type for the user and an obesity probability projection for the user;
in accordance with the determined latent obesity type and the obesity probability projection, generating an obesity mitigation action plan with a plurality of steps; and
a display configured to display the action plan on the display.

11. The computing device of claim 10, wherein the health characteristics further includes current physical environment and current lifestyle of the user.

12. The computing device of claim 11, wherein the one or more programs further include instructions for:
in accordance with the computed orientation metrics and a first set of the computed health metrics corresponding to the current physical environment and current lifestyle of the user, determining one or more obesity inducing stress markers for the user.

13. The computing device of claim 12, wherein generating the obesity mitigation action plan includes utilizing the determined obesity inducing stress markers for the user.

14. The computing device of claim 13, wherein the one or more programs further include instructions for:
in accordance with the determined obesity inducing stress markers and a second set of the computed health metrics corresponding to the personal health history and the awareness of personal health, determining an obesity incidence precursor projection for the user;
wherein the obesity probability projection for the user is further determined based on the determined obesity incidence precursor projection.

15. The computing device of claim 13, wherein the determined obesity probability projection represents a probability of whether obesity will occur within a predetermined span of time.

16. The computing device of claim 10, wherein at least one of the plurality of steps includes receiving user feedback in response to the action plan.

17. The computing device of claim 10, wherein at least one of the plurality of steps includes providing exercise instructions for the user to follow.

18. The computing device of claim 10, wherein the latent obesity type belongs to a category that represents a particular physical engagement level, a particular cognitive engagement level, and a particular behavioral engagement level derived from the computed orientation metrics.
